(19) United States
(12) Patent Application Publication
(10) Pub. No.: US 2009/0157108 A1
(43) Pub. Date: Jun. 18, 2009

(54) MICROKERATOME CUTTING BLADE

(76) Inventor: Randy Dame, High Ridge, MO (US)

Correspondence Address: Bausch & Lomb Incorporated, One Bausch & Lomb Place, Rochester, NY, 14604-2701, US

(21) Appl. No.: 11/955,632

(22) Filed: Dec. 13, 2007

(51) Int. Cl. A61F 9/007

(52) U.S. Cl. 606/166

(57) ABSTRACT

A microkeratome cutting-blade assembly 10 includes a cutting-blade 12 having a cutting edge 22 and apertures 18 within the blade 12. Each aperture 18 includes an inwardly-extending portion 20 on the side of the aperture 18 closest to the cutting edge 22. The assembly 10 includes a blade holder 14 having post members 16 configured to be received within the apertures 18. The cutting-blade 12 is attached to the blade holder 14 by staking the post members 16 received within the apertures 18. The cutting-blade's inwardly extending portions 20 cause the cutting-blade 12 to be maintained in accurate alignment while cold staking the post members 16 to expand and fill the apertures 18.

### FIELD

The present invention relates to cutting-blade assemblies and specifically, cutting-blade assemblies for use in a microkeratome for use in ophthalmic surgery.

### BACKGROUND

The statements in this section merely provide background information related to the present disclosure and may not constitute prior art.

Laser-Assisted In-situ Keratomileusis or LASIK surgery has become a widespread and effective eye correction surgical procedure in the last several years. Before a laser ablates a portion of a patient's corneal tissue to correct that patient's vision, a flap of the patient's cornea must be formed.

A typical cornea, on average, is about 520 microns thick. A typical flap thickness for the corneal flap, that is formed prior to laser ablation and LASIK surgery, is desired to be on the order of 160 to 200 microns. As is well known, these corneal flaps are made using microkeratomes that travel in a linear, arcuate, or even in a horizontally hinged path. A microkeratome typically cuts the corneal flap using a cutting-blade assembly made with standard razor blade stock available from any of numerous razor blade manufacturers, though other materials such as ceramics or plastics may be used. It is also typical that the cutting-blade is oscillated to aid in the cutting, while the cutting-blade is translated across the cornea to form a corneal flap.

A rather accurate measurement of the corneal thickness prior to LASIK surgery is obtainable through any number of known measurement methods, such as the use of an Orbscan™ Topography System available from Bausch & Lomb Incorporated. After the corneal thickness measurement has been obtained, depending on the surgeon's preference and the amount of correction needed, a flap thickness determination is then chosen by the surgeon.

Typically, in the prior art, each microkeratome comes with a variety of cutting heads, which are precisely manufactured to obtain different flap thicknesses, such as cuts of 160 microns, 180 microns, and 200 microns. Again, in the prior art, a single cutting-blade assembly has been used with these different precision cutting heads to obtain the different flap thicknesses.

One variation to this is from Med-Logics, Inc, Med-Logics currently manufactures LASIK blades, which consist of a piano or nominal length blade and a plus and a minus blade, wherein the blade extensions vary from the piano extension either plus or minus 20 microns. According to Med-Logics, this then allows the doctor to produce a flap of thinner or thicker thickness from the piano blade using a given cutting head.

A problem with all prior art microkeratome cutting-blade assemblies has been the consistency of the blade extension of the cutting head of the cutting-blade assembly. The blade extension is defined as the distance from the cutting tip of the blade to the nearest point of the blade holder. A microkeratome cutting head is precisely machined to applanate the cornea a given amount and to hold the blade holder within fairly tight tolerances. Many attempts and improvements to control blade extension and hence flap thickness have been made. It has always been a goal to provide a consistent and predictable flap thickness with a given cutting-blade in a given microkeratome cutting head.

The consistency of the flap thickness cut is important in order to reduce the amount of variance from cut-to-cut so that more consistent, predictable cuts may be made.

While it is possible to obtain a corneal thickness measurement before LASIK surgery, it has proven extremely difficult to measure corneal thickness of an eye with a corneal flap laid back over, and it is equally difficult to obtain a reliable corneal flap thickness measurement due to changes in hydration of the corneal flap and the cornea which occur quite rapidly under the surgical lights of an operating room.

Therefore, it is desirable to provide a microkeratome cutting-blade assembly having a tightly controlled blade extension and to provide an easily accomplished method of producing such a tight blade extension.

It has been found that attaching a blade holder to a cutting-blade by known methods such as cold staking, heat staking, or adhesive bonding provide a robust bond to maintain the precise blade extensions desired under certain circumstances, but it would be desirable to provide an attachment between the blade holder and cutting-blade that is robust and aids in achieving a tight blade extension tolerance but yet economical to manufacture.

### SUMMARY

The present disclosure relates to microkeratome cutting equipment for applanating the cornea, in which a microkeratome cutting blade is provided. One embodiment of a microkeratome cutting blade assembly is provided that includes a cutting-blade having a cutting edge and apertures within the blade. Each aperture includes an inwardly-extending portion on the side of the aperture closest to the cutting edge. The assembly includes a blade holder having post members configured to be received within the apertures. The cutting-blade is attached to the blade holder by staking the post members received within the apertures. The cutting-blade's inwardly extending portion tends to cause the cutting-blade to be maintained in accurate alignment while cold staking the post members to expand and fill the apertures.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a perspective view of a cutting-blade assembly in accordance with the present disclosure;

FIG. 2 is a bottom view of a cutting-blade in accordance with the present disclosure; and

FIG. 3 is a bottom view of an alternate embodiment of a cutting-blade in accordance with the present invention.

### DETAILED DESCRIPTION

The following description is merely exemplary in nature and is not intended to limit the present disclosure, application, or uses. It should be understood that throughout the drawings, corresponding reference numerals indicate like or corresponding parts and features.

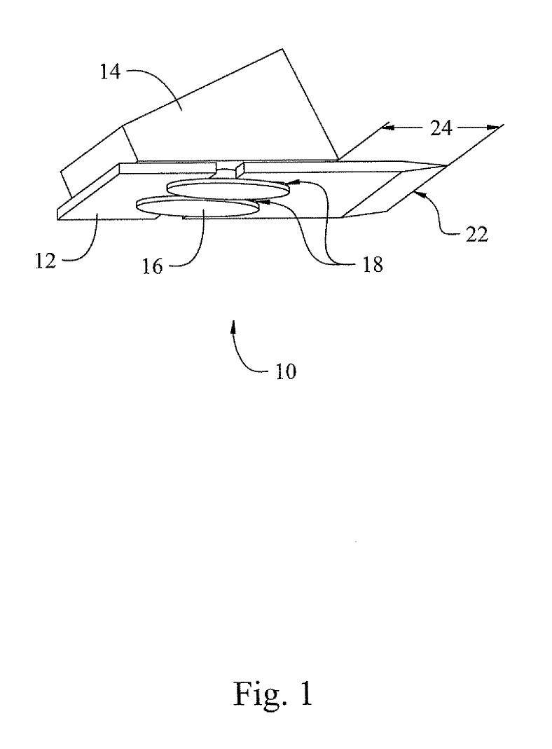

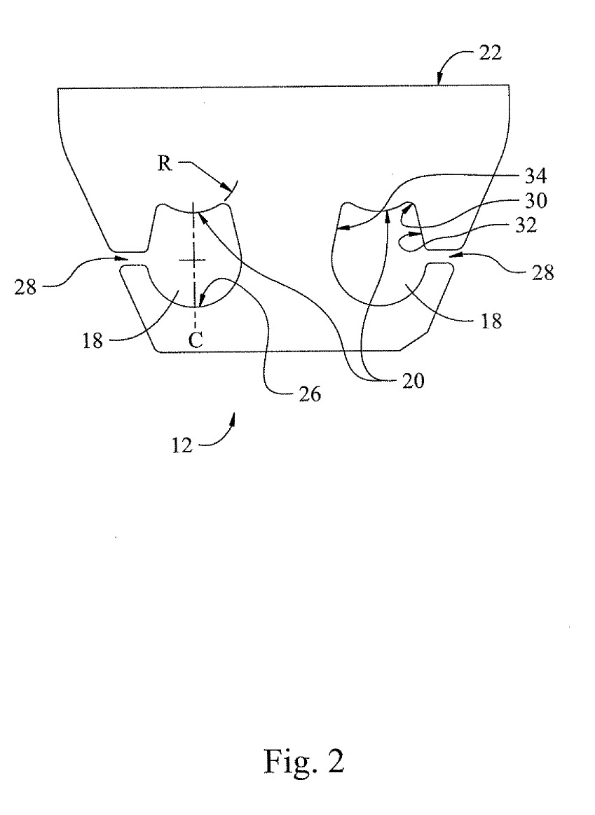

FIG. 1 shows a microkeratome cutting-blade assembly 10, in accordance with the present invention. Assembly 10 includes a cutting-blade 12 and a blade holder 14 attached to the cutting-blade 12. Preferably, blade holder 14 is attached to cutting-blade 12 via post members 16 that extend through apertures 18 in cutting-blade 12. The apertures 18 in the cutting-blade 18 are generally through-holes that include inwardly-extending portions 20 (shown in FIG. 2) on the side of the aperture closest to the cutting edge 22 of the cutting-blade 12.

To assemble the cutting-blade to the holder 14, the post members 16 are secured within the apertures through a commonly known procedure such as heat staking, cold staking, or other means. Preferably, a blade extension length represented by number 24 is critical to use of the blade, and is controlled to within a tight tolerance of a target extension length. Maintaining this target blade extension length is critical to providing a consistent, predictable corneal-flap thickness. Blade extension 24 may also be measured from a front surface of holder 14 to a line parallel to the front surface and passing through the cutting tip of blade 12.

Before staking, some voids or gaps may exist between the blade holder 14 and cutting-blade 12. These gaps are a by-product of achieving the desired target blade extension tolerances. This is because the post member 16 must be moveable within the through-hole so that the cutting-blade can be positioned to achieve a precise blade extension length 24. For example, the cutting blade 12 may first be positioned over the post members 16 of a blade holder 14 being held in a fixture, and then moved to place the cutting edge 22 against a stop in the fixture, for positioning the cutting blade 12 to achieve a desired blade extension length 24. However, there may not be enough post member material that is displaced during cold staking to fill all the gaps and maintain the blade 12 at the precise blade extension length 24.

Referring to FIG. 2, the cutting-blade's apertures 18 preferably include inwardly-extending portions 20 that assist in maintaining the blade-extension length 24. The apertures 18 in the cutting-blade 18 are generally through-holes that include inwardly-extending portions 20 on the side of the aperture closest to the cutting edge 22 of the cutting-blade 12. However, the apertures 18 may further include notches 28 formed by electro-deposition machining of apertures 18, as shown in FIG. 2. The inwardly extending portions 20 of the cutting-blade 12 extend into the opening area of the apertures 18 on the side closest to the cutting edge 22. The inwardly extending portions 20 form a generally curved contour, which has an orientation that is symmetrical about the centerline “C”, of the aperture 18, and generally parallel to the cutting edge 22. As the cold staking of the post members 14 occurs, the inwardly extending portions 20 assist in causing the cutting-blade 12 to be pushed or maintained against a stop, for setting the blade's extension length 24. Preferably, the inwardly extending portion defines an arcuate edge 20 that protrudes or extends inwardly into the opening area of the through-hole 18. The staking of the post members 16 causes the post member material to expand, and thereby contact the lower edge 26 of the aperture 18 and the upper arcuate edge 20. The contact between the expanding post material and the arcuate edge 20 causes the cutting-blade 12 to be pushed in a direction that will tend maintain the cutting-blade's cutting edge 22 in a position for achieving a desired extension length 24. For example, the cutting blade 12 may be positioned over the post members 16 of a blade holder 14 held in a fixture, and moved in a direction for positioning the cutting edge 22 against a stop in the fixture at which the cutting edge 22 is at a desired extension length 24.

FIG. 2 is a bottom view of the assembly 10 of FIG. 1. The blade may further include notches 30 between the inwardly extending portion 20 and the remaining periphery of the aperture 18. The purpose of notches 30 is to allow the material of post 16 upon staking to fill the notches 30 and ensure secure attachment of the blade 12 to the blade holder 14. However, it may be preferable not to form notches 30 in blade 12. Likewise, the cutting-blade's apertures 18 may further include tapered sides 32 and 34, as shown in FIG. 2. Preferably, blade holder 14 is made of Lubiloy™ and is molded or machined. Lubiloy™ is a polycarbonate material, which is preferred for blade holder 14, though any known suitable material is acceptable for blade holder 14, such as Delrin™. As previously discussed, cutting-blade 12 is preferably formed from razor blade stock widely available from a number of manufacturers, although a number of other materials are also possible.

A gap must exist between the post 16 and through-holes 18 in cutting-blade 12 to allow for positioning the blade extension on the blade holder in assembly. During assembly, the holder 14 moves slightly relative to the blade 12 so that the desired blade extension position can be established before staking the holder 14 to the blade 12. It has been found that because of the necessary gap between the post members 16 and cutting-blade 12, the cutting blade 12 may not be reliably maintained in accurate alignment while cold staking causes the post material to fill up the gap. It has been found that the inwardly extending portion 20 that extends into the opening area of the aperture 18 assists in causing the blade 12 to be maintained in alignment during the cold staking process. In this way the tight blade extension tolerances desired may be maintained throughout the staking process and operation or use of the cutting-blade assemblies.

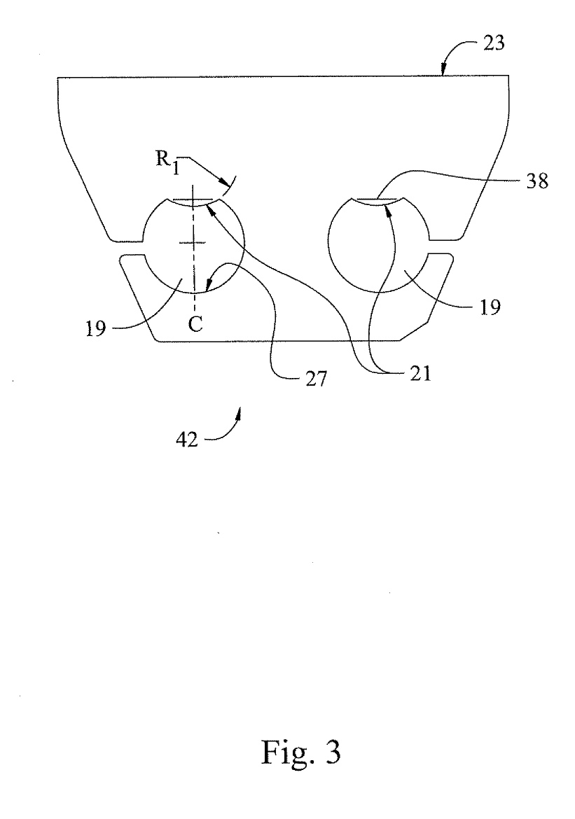

Referring to FIG. 3, an alternative embodiment is shown of a microkeratome cutting-blade 42, in accordance with the present disclosure. The blade 42 is preferably connected to a blade holder (not shown) via post members that are configured to be received within apertures 19 of the blade, and cold-staked. The blade 42 includes apertures 19 having inwardly-extending portions 21 that assist in maintaining a blade-extension length. The apertures 19 in the cutting-blade 42 are generally through-holes that include inwardly-extending portions 21 on the side of the aperture closest to the cutting edge 23 of the cutting-blade 42. The inwardly extending portions 21 of the cutting-blade 42 extend into the opening area of the apertures 19 on the side closest to the cutting edge 23. The inwardly extending portions 21 form a generally curved contour, which has an orientation that is symmetrical about the centerline “C” of the aperture 19, and generally parallel to the cutting edge 23. When cold staking of a post member received within aperture 19 occurs, the inwardly extending portions 21 assist in causing the cutting-blade 42 to be pushed or maintained against a stop, for setting the blade's extension length. Preferably, the inwardly extending portion defines an arc having a radius “R₁” extending along a secant line 38 that is parallel to the cutting edge 23 and intersects the through-hole 19 on the side of the through-hole 19 closest to the cutting edge 23. The arc extends along the length of the secant line 38 to define an arcuate edge 21 that protrudes or extends inwardly into the opening area of the through-hole 19. The staking of post members received within the apertures 19 causes the post member material to expand, and contact the lower edge 27 of the aperture 19 and the upper arcuate edge 21 that extends into the aperture 19. The contact between expanding post material and the arcuate edge 21 causes the cutting-blade 42 to be pushed in a direction that will maintain the cutting-blade's cutting edge 22 in a position for achieving a desired extension length. For example, the cutting blade 42 may be positioned over the post members of a blade holder being held in a fixture, where the cutting blade 42 is moved in a direction towards a stop in the fixture to position the cutting edge 23 of the cutting blade 42 at a desired extension length.

In another aspect of the present disclosure, a method of forming a microkeratome cutting-blade assembly is provided. The method comprises forming a microkeratome cutting-blade assembly comprises the steps of positioning a cutting blade, which has apertures that include inwardly-extending portions on the aperture side closest to the blade's cutting edge, onto a blade holder having post members configured to be received within said apertures. The method further includes the steps of moving the cutting blade relative to said blade holder to cause said cutting edge to contact a stop in a fixture (not shown) in which said blade holder is held, and staking the cutting-blade to the blade holder to form a cutting-blade assembly, wherein the staking causes the post members to expand and fill gaps between the blade holder's post members and the cutting-blade's apertures.

From the above, it may be appreciated that the present invention provides an improvement to microkeratome cutting-blades and assemblies thereof. The present invention is illustrated herein by example, and various modifications may be made by a person of ordinary skill in the art. While the inwardly extending portions have been shown as curved or radial, other geometries are possible, such as triangular or octagonal shaped portions.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A microkeratome cutting-blade assembly comprising:
a cutting-blade having a cutting edge and apertures within the blade, each aperture including an inwardly-extending portion on the side of the aperture closest to the cutting edge;
a blade holder having post members configured to be received within said apertures; and
wherein the cutting-blade is attached to the blade holder by staking the post members received within the apertures.

2. The microkeratome cutting-blade assembly of claim 1, wherein the inwardly-extending portions assist in maintaining a desired blade-extension length during cold staking of the post member.

3. The microkeratome cutting-blade assembly of claim 2 wherein the inwardly extending portion causes the cutting-blade to be maintained in accurate alignment while cold staking the post members to expand and fill the apertures.

4. The microkeratome cutting-blade assembly of claim 1, wherein the inwardly extending portions form a generally curved contour that is symmetrically oriented relative to the aperture and generally parallel to the cutting edge.

5. The microkeratome cutting-blade assembly of claim 1, wherein the inwardly extending portion defines an arcuate edge along a secant line that is parallel to the cutting edge and intersects the through-hole on the side of the through-hole closest to the cutting edge.

6. The microkeratome cutting-blade assembly of claim 5, wherein the contact between the expanding post material and the arcuate edge causes the cutting-blade to be pushed in a direction that will maintain the cutting-blade's cutting edge in a position for achieving a desired extension length.

7. The microkeratome cutting-blade assembly of claim 1 wherein the inwardly extending arcuate edge causes the cutting-blade to be maintained in accurate alignment while cold staking causes the post members to expand and fill the apertures.

8. The microkeratome cutting-blade assembly of claim 7, wherein the inwardly extending portions are configured to cause the cutting-blade to be moved in a direction against a stop of a fixture during staking of the post members, to assist in positioning the cutting edge of the cutting blade at a desired extension length.

9. The microkeratome cutting-blade assembly of claim 1, wherein the cutting blade is secured to the blade holder by one of heat staking or cold staking of the post members, to cause the post member material to expand within the apertures.

10. A microkeratome cutting-blade comprising:
a cutting-blade having a cutting edge thereon, and a plurality of apertures within the blade, each aperture including inwardly-extending portions on the side of the aperture closest to the cutting edge, wherein each aperture is configured to receive a post member of a blade holder therein.

11. The microkeratome cutting-blade of claim 10, wherein the inwardly extending portions form a generally curved contour that is symmetrically oriented relative to the aperture and generally parallel to the cutting edge.

12. The microkeratome cutting-blade of claim 10 wherein the inwardly extending arcuate edge causes the cutting-blade to be maintained in accurate alignment while cold staking causes the post members to expand and fill the apertures.

13. The microkeratome cutting-blade of claim 10, wherein the inwardly extending portions are configured to cause the cutting-blade to be moved in a direction against a stop of a fixture during staking of the post members, to assist in positioning the cutting edge of the cutting blade at a desired extension length.

14. The microkeratome cutting-blade of claim 13, wherein the inwardly extending portion defines an arcuate edge along a secant line that is parallel to the cutting edge and intersects the through-hole on the side of the through-hole closest to the cutting edge.

15. A method of forming a microkeratome cutting-blade assembly comprising the steps of:
positioning a cutting blade, which has apertures that include inwardly-extending portions on the aperture side closest to the blade's cutting edge, onto a blade holder having post members configured to be received within said apertures; and
staking the cutting-blade to the blade holder to form a cutting-blade assembly, wherein the staking causes the post members to expand and fill gaps between the blade holder's post members and the cutting-blade's apertures.

16. The method of claim 15 wherein the inwardly extending portion causes the cutting-blade to be maintained in accurate alignment while cold staking causes the post members to expand and fill the apertures.

17. The method of claim 15 wherein the inwardly extending portions are configured to cause the cutting-blade to be moved in a direction against a stop of a fixture during staking of the post members, to assist in positioning the cutting edge of the cutting blade at a desired extension length.

18. The method of claim 15 wherein the inwardly extending portion defines an arcuate edge that intersects the through-hole on the side of the through-hole closest to the cutting edge, and wherein the contact between the expanding post material and the arcuate edge causes the cutting-blade to be pushed in a direction that will maintain the cutting-blade's cutting edge in a position for achieving a desired extension length.

19. The method of claim 15 wherein the cutting blade is secured to the blade holder by one of heat staking or cold staking of the post members, to cause the post member material to expand within the apertures.
